(19) United States
(12) Patent Application Publication
(10) Pub. No.: US 2007/0298045 A1
(43) Pub. Date: Dec. 27, 2007

(54) Method and vaccine for non-genetically engineered atavism growth enhancement

(75) Inventor: Bor-Rung Ou, Taichung City (TW)

Correspondence Address: Genereach Biotechnology Corporation, P.O. Box No. 6-57, Junghe, Taipei, 235, omitted

(73) Assignee: Genereach Biotechnology Corporation

(21) Appl. No.: 11/812,274

(22) Filed: Jun. 18, 2007

(30) Foreign Application Priority Data
Jun. 22, 2006 (TW) 095122429

(51) Int. Cl. A61K 39/395; A61P 43/00

(52) U.S. Cl. 424/178.1

(57) ABSTRACT

A method and vaccine for non-genetically engineered atavism growth enhancement, which injects the vaccine with myostatin antibody containing muscular tissues into the vertebrate mother's body prior to the pregnancy stage; the genes of myostatin that contain muscular tissues can enhance the muscular mass and growth rate of the muscle.

### BACKGROUND OF THE INVENTION

(a) Field of the Invention

The present invention relates generally to a method for growth enhancement and its vaccine applicable on vertebrates, and more particularly to a method and vaccine for non-genetically engineered atavism growth enhancement.

(b) Description of the Prior Art

Pig breeding in Taiwan is highly affected by its damp, hot climatic conditions and disease problems. Notwithstanding little domestic researches on lean growth modes, some foreign countries have shifted their focus from daily gain and feed conversion to lean gain and lean efficiency for an improved meat quality and eating quality.

Though the meat quality in Taiwan is always believed to be superior to that in other countries, this argument is not yet demonstrated by any research or production data so far. However, the research on meat quality and eating quality of Taiwanese boar family was completed in the U.S. between 1991-1994. Meanwhile, the research on sow family is expected to be finished in 2001. The research on the meat quality of Taiwanese boar—involving over 200 items—was completed in Canada in 1997; Denmark implemented a 4-year-long integrated plan on the meat quality starting from 1995. All these researches show that meat quality and eating quality are key economic indices in the next century, which place a high demand on Taiwan livestock industry.

On the other hand, improper or excessive application of hormone and antibiotics in livestock industry make the customers vulnerable to cancer hazard. And, there is no other evidence showing if meat from gene engineering leads to any squeal or other negative impact on human body. So, the meat quality shall be improved, while the muscle growth rate and muscular mass are also a major factor to cost reduction for this industry.

### SUMMARY OF THE INVENTION

The present invention proposes a method and vaccine for non-genetically engineered atavism growth enhancement, in lieu of gene engineering, gene modification or utilization of antibiotics and hormone. This helps to improve the offspring's muscle growth rate and muscular mass, thus improving efficiently the meat quality and sharpening the competitive edge of livestock Industry.

A method and vaccine for non-genetically engineered atavism growth enhancement, which injects the vaccine with myostatin antibody containing muscular tissues into the vertebrate mother's body prior to the pregnancy stage; the genes of myostatin that contain muscular tissues can enhance the muscular mass and growth rate of the muscle. The details and technology of the present invention are described below with reference to the accompanying drawings:

### BRIEF DESCRIPTION OF THE DRAWINGS

FIG. 1 is a flow diagram of the present invention in accordance with the method for non-genetically engineered atavism growth enhancement.

FIG. 2 is a schematic drawing of the present invention in accordance with the extraction formula for non-genetically engineered atavism growth enhancement.

FIG. 3 is a curve diagram of the present invention in accordance with the male test method for non-genetically engineered atavism growth enhancement.

FIG. 4 is a curve diagram of the present invention in accordance with the female test method for non-genetically engineered atavism growth enhancement.

### DETAILED DESCRIPTION OF THE PREFERRED EMBODIMENTS

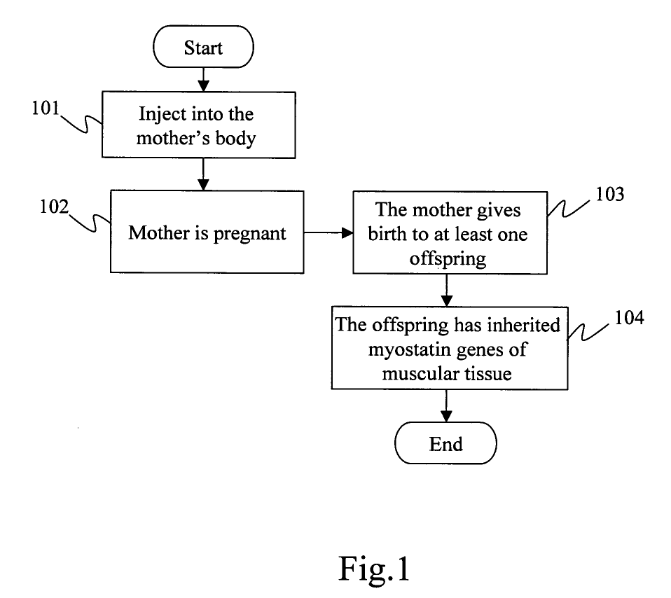

The present invention relates generally to a method for growth enhancement and its vaccine applicable on vertebrates, as shown in FIG. 1. The major purpose of the present invention is to enhance muscular mass and growth rate of muscle. Firstly, inject vaccine into vertebrate mother's body (step 101). The vaccine must contain myostatin antibody of muscular tissues. Then, the mother is pregnant (step 102), in other words, the mother's body is injected with vaccine prior to pregnancy, but also immune to vaccine. Next, the mother's body gives birth to offspring (step 103), with the number of offspring depending upon the species group of mother. For example, a mother pig (sow) often gives birth to 2-6 piglets that contain the genes of myostatin of muscular tissue (step 104). This could increase considerably muscular mass and improve the growth rate and meat quality, thus making great contribution to the livestock industry.

Myostatin is a negative regulation factor of muscular tissue. As a typical example, the mutation of the genes of Myostatin once led to numerous muscular tissue of “double-muscled” Belgian blue cattle. If monoclonal antibody of an anti-Myostatin is used to blockade the activity of Myostatin in mdx mouse sample suffering from Duchenne's muscular dystrophy, muscular tissues could be increased and improved dramatically. Myostatin blockade is an attractive solution for muscular dystrophy, namely, Myostatin is currently applied to cure muscular dystrophy in the medical field.

Hence, Myostatin (MSTN; Gdf-8) is likely a major regulation factor for muscular development and growth of higher vertebrate. It is proved that “Double-muscled” Belgian blue cattle are related to the deletion, transition or transversion of protein code sequence of myostatin genes. The mutein of myostatin gene leads to rapid growth of muscle after hyperplasia and hypertrophic.

Muscle retardant is an important cell factor to control muscular development and growth. The mononucleotide polymorphism of Myostatin genes was detected by means of PCR-SSCP and DNA. And, mononucleotide polymorphism of Myostatin was also analyzed in relation to slaughtering weight, breast muscle weight, leg muscle weight, liver weight and abdominal fat weight. The results show that, P60/61 gene (AA, BB and AB) has influence upon 12-years old abdominal fat weight, abdominal fat rate, birth weight and breast muscle rate (P<0.05): abdominal fat weight of AA is much higher than BB (P<0.05); abdominal fat rate of AA and AB is much higher than BB (P<0.05); birth weight of AA is much higher than BB (P<0.05); breast muscle rate of AA is much higher than AB (P<0.05). P80/P81 gene (CC, DD and CD) is related to breast muscle weight (P<0.05) and breast muscle rate (P<0.01): there is a significant difference of breast muscle weight between CC and DD (P<0.05), of which CC has a bigger breast muscle weight; there is an extremely significant difference of breast muscle rate between CC and DD (P<0.01), and a significant difference between CC and CD (P<0.05), of which CC has a higher breast muscle rate than CD and DD, and CD has also a higher breast muscle rate than DD(P<0.05). P93/94 gene (EF and EE) is closely related to breast muscle rate (P<0.05): EF has a higher breast muscle rate than EE. It is proved that Myostatin genes are related to muscular development and growth, and more importantly involved in fat metabolism and deposit.

Therefore, the zoologists and geneticists of ARS (Agricultural Research Service) now make efforts to study an approach to increase lean beef from cattle genes. If the genes for the protein are changed or restrained, it is possible to facilitate muscular growth and reduce the fat storage. Traditionally, the animal breeders increase the lean beef through a selection plan. According to the findings of Meat Animal Center (MARC) in Nebraska, restraining myostatin protein within cattle could produce flexible and low-content saturated fatty acid.

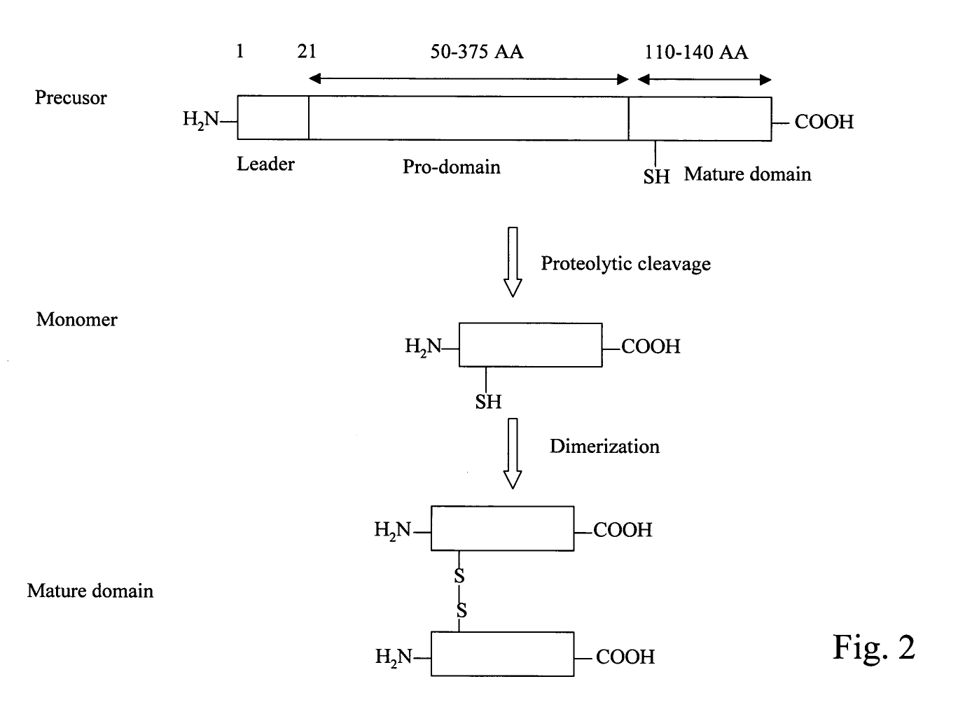

FIG. 2 depicts a formula of the present invention for non-genetically engineered atavism growth enhancement. The monomer required by Precusor is extracted through Proteolytic cleavage, and then allowed for Dimerization to form the vaccine. Therefore, the vaccine contains myostatin antibody that has no influence upon mother's body, nor changes its gene. Meanwhile, the mother's body does not embody the myostatin gene of muscular tissue. Instead, only the offspring has such performance by means of atavism.

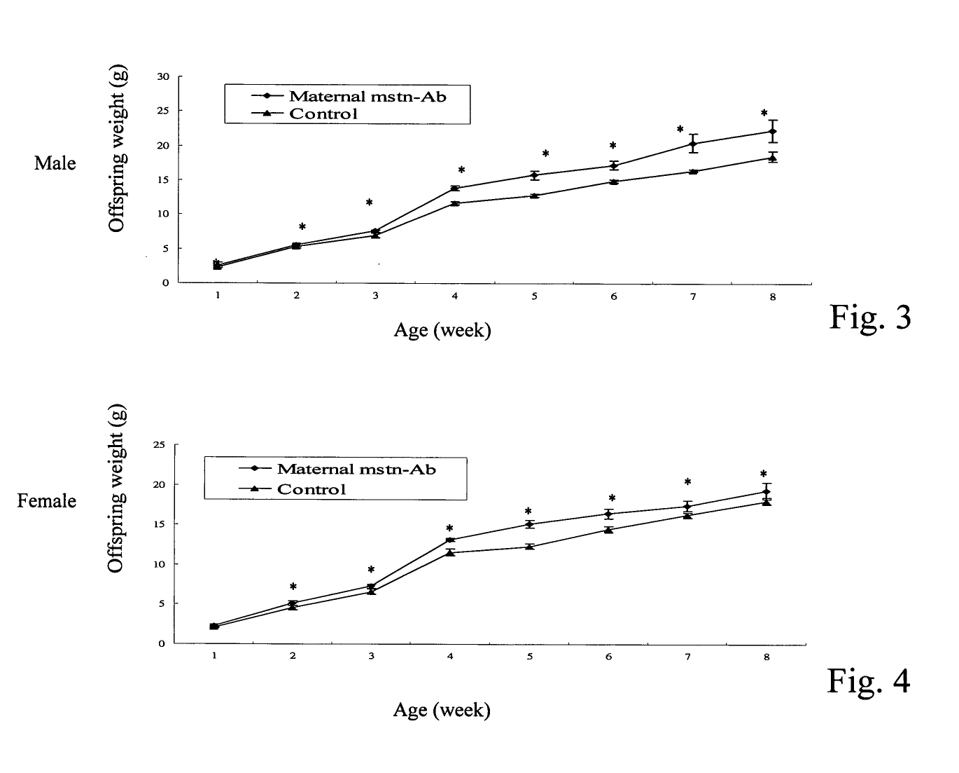

Referring to FIG. 3 and FIG. 4, this formula could greatly improve the weight of male and female offspring irrespective of the gender.

Thus, the present invention proposes a method and vaccine for non-genetically engineered atavism growth enhancement, in lieu of gene engineering, gene modification or utilization of antibiotics and hormone. This helps to improve the offspring's muscle growth rate and muscular mass, thus improving efficiently the meat quality and sharpening the competitive edge of livestock Industry.

Although the invention has been explained in relation to its preferred embodiment, it is to be understood that many other possible modifications and variations can be made without departing from the spirit and scope of the invention as hereinafter claimed.

What is claimed is:

1. A method for non-genetically engineered atavism growth enhancement, which comprising the following steps:
inject the vaccine into the vertebrate mother's body; the vaccine contains myostatin antibody containing muscular tissues, which has no influence upon the growth of mother's body;
the mother is pregnant;
the mother gives birth to at least an offspring; and
the offspring has inherited myostatin genes of muscular tissue that could increase the muscular mass and growth rate.

2. The method as defined in claim 1, wherein the vaccine is injected into mother's body prior to pregnancy.

3. The method as defined in claim 1, wherein the antibody has no influence upon mother's body, nor changes its gene.

4. The method as defined in claim 3, wherein the antibody does not let the mother's body embody the myostatin gene of muscular tissue.

5. A vaccine for non-genetically engineered atavism growth enhancement, which is injected into vertebrate mother's body, such that the mother's body gives birth to an offspring inheriting myostatin gene containing muscular tissue; the muscular mass and growth rate is increased; the vaccine comprises:
a myostatin antibody containing muscular tissue, which has no influence upon the growth of mother's body, and is injected into the mother's body prior to the pregnancy stage; the offspring inherits myostatin genes of muscular tissue to increase its muscular mass and growth rate.

6. The vaccine as defined in claim 5, wherein the antibody has no influence upon the mother's body, nor changes its gene.

7. The vaccine as defined in claim 6, wherein the antibody does not let the mother's body embody the myostatin gene of muscular tissue.
